(19) United States
(12) Patent Application Publication
(10) Pub. No.: US 2008/0020376 A1
(43) Pub. Date: Jan. 24, 2008

(54) Enriched Cdna Expression Libraries and Methods of Making and Using Same

(76) Inventors: Armen N. Akopian, San Antonio, TX (US); Christopher M. Flores, Lansdale, PA (US)

Correspondence Address: NEEDLE & ROSENBERG, P.C., SUITE 1000, 999 PEACHTREE STREET, ATLANTA, GA, 30309-3915, US

(21) Appl. No.: 10/591,892

(22) PCT Filed: Mar. 9, 2005
(86) PCT No.: PCT/US05/08064

§ 371 Date: Jun. 4, 2007

Related U.S. Application Data
(60) Provisional application No. 60/551,741, filed on Mar. 10, 2004.

(51) Int. Cl. C12Q 1/68; C07H 21/04; G01N 33/53; G01N 33/566

(52) U.S. Cl. 435/6; 435/7.2; 436/501; 536/24.1; 536/25.3

(57) ABSTRACT

Disclosed are cDNA expression libraries enriched for cDNAs that encode, for example, secretory and membrane-bound proteins and to methods for making such libraries. Also disclosed are methods for using the cDNA expression libraries disclosed herein to screen for proteins, nucleic acids, and agents that modulate the function and/or expression of proteins.

### CROSS-REFERENCE TO RELATED APPLICATIONS

This application claims priority to U.S. Application No. 60/551,741, filed Mar. 10, 2004. The aforementioned application is hereby incorporated herein by reference in its entirety.

### STATEMENT REGARDING FEDERALLY SPONSORED RESEARCH

The research described herein was supported by the National Institute on, Drug Abuse (Grant Number DA 11959). The U.S. Government has certain rights in this invention.

### FIELD

The disclosed subject matter relates to cDNA expression libraries, methods for their preparation, and methods for their use. Specifically, the disclosed subject matter relates to cDNA expression libraries enriched for membrane-bound polysomal mRNA.

### BACKGROUND

Expression cloning, which allows for the isolation of genes of interest based on their functional properties, is one of the most powerful approaches in molecular biology. Generally, expression cloning involves generating a cDNA expression library and incorporating the cDNA from that library into expression vectors. The vectors are then transfected into a cell population, which expresses the product encoded by the cDNA. Using assays that screen for a particular gene product(s) (e.g., a protein), cells that express the desired gene product(s) can be identified. cDNA from these identified cells can then be isolated, purified, and cloned.

Despite the inherent benefits of this approach, such as its applicability to many different cell types, genes, and gene products, expression cloning still remains one of the least used approaches for gene/protein isolation. A major obstacle for the broader employment of expression cloning is its low sensitivity. Accordingly, there has been a great deal of research in the area of expression cloning and for ways to improve the sensitivity of such methods.

Some efforts to improve the sensitivity of expression cloning focus on the preparation and use of enriched cDNA libraries, i.e., libraries in which there is a greater concentration of cDNA for a gene product(s) of interest and/or a lesser concentration of cDNA for an undesired gene product(s) than is typically observed in a cDNA library prepared by conventional methods. To this end, researchers have sought to prepare enriched cDNA expression libraries from mRNA that is itself enriched, that is, mRNA that contains a greater concentration of mRNA encoding a gene product(s) of interest and/or a lesser concentration of mRNA encoding an undesired gene product(s) than is typically observed in mRNA obtained by conventional methods. In practice, methods of obtaining enriched mRNA basically aim at isolating cellular fractions that only contain desired levels and/or types of mRNA. However, such methods have been fraught with challenges and problems, such as contamination and inadequate isolation.

It was generally thought, following one of the dogmas of molecular biology, that a gene is expressed (i.e., translated into protein) as soon as primary transcript is transported to the cytoplasm. However, increasing amounts of evidence indicate that many mRNAs are subject to translational control on different levels. For example, soluble cytoplasmic proteins are synthesized on free ribosomes, whereas secretory and integral membrane proteins are synthesized on membrane-bound polysomes. Accordingly, a cell's cytoplasmic and membrane fractions can be generally characterized as having different types and/or amounts of mRNAs. Moreover, similar cell fractions from different cell types can also contain different amounts of mRNA and ribosomes. For example, while membrane-bound polysomes are present in all nucleated cells except sperm, they are especially abundant in cells engaged in protein secretion or extensive membrane-protein synthesis (Andrews and Tata, Biochem J (1972) 127(2):6P). Neurons are but one example of cells belonging to the category of cells that extensively produce proteins both for secretion and for control of neuron excitability (e.g., ion channels). Thus, the purification of, for example, membrane-bound polysomes can be a useful enrichment step in the cloning of specialized receptors, channels, and secretory proteins and their further characterization.

Basic methods for separating free ribosomes from membrane-bound polysomes were developed in the 60's and 70's (Blobel and Potter, J Mol Biol (1967) 26(2):279-292; Venlkatesan and Steele, Biochim Biophys Acta (1972) 287(3):526-537; Ramsey and Steele, Anal Biochem (1979) 92(2):305-313). According to these methods, separation is achieved by exploiting either the difference in density or size between free ribosomes and rough microsomes, which contain membrane-bound polysomes. Since these early beginnings, researchers have been studying the many factors influencing the efficiency of these separations.

Methods that have been developed for isolating membrane-bound polysomes, which very much resemble each other, do not possess enough efficiency or sensitivity to isolate moderately or rarely represented transcripts of, for example, receptors and channels. Such inadequacies can be generally explained by dividing all mRNA into three categories: (1) mRNAs that are not associated with ribosomes (free-ribo-protein particles); (2) mRNAs that are associated with free polysomes; and (3) mRNAs that are associated with membrane-bound polysomes. Membrane-bound polysomes can in turn be classified into polysomes that are: (a) loosely bound to the endoplasmic reticulum or (b) tightly bound to the endoplasmic reticulum. The original methods for the isolation of membrane-bound polysomes did not verify the completeness of separation of membrane-bound polysomes from free ribosomes (Blobel and Potter, J Mol Biol (1967) 26:279-292). However, it later became apparent that loosely bound polysomes and free polysomes share many common properties (Ramsey and Steel, Biochem J (1977) 168:1-8). As such, previously described methods for the isolation of membrane-bound polysomes are not adequate; that is, membrane-bound polysomes isolated by these methods are contaminated with free polysomes. Thus, a substantial amount of cyclophilin, which is synthesized on free-ribosomes, is still present in a membrane-bound polysome fraction isolated by typical methods.

Therefore, there is currently a need for methods of constructing cDNA expression libraries (i.e., cDNA libraries constructed in an expression/shuttle vector), which increase the sensitivity of expression cloning methods for the isolation of genes encoding, for example, trans-membrane and secretory proteins. The materials, compositions, methods, articles, and devices disclosed herein meet this need.

### SUMMARY

In accordance with the purposes of the disclosed materials, compositions, methods, articles, and devices, as embodied and broadly described herein, in one aspect, the disclosed subject matter relates to cDNA expression libraries enriched for cDNA that encode, for example, secretory or membrane-bound proteins and to methods for making such libraries. Also described herein are methods for using the cDNA expression libraries disclosed herein to screen for nucleic acids, proteins, and agents that modulate biologic activities, including, for example, function or expression.

Additional advantages will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF DRAWINGS

The accompanying drawings, which are incorporated in and constitute a part of this specification, illustrate several aspects described below.

FIG. 1 is a two-paneled photograph from a Northern blot analysis for various mRNA isolation and purification conditions. The conditions under which the mRNA was isolated are indicated above each lane. The top panel is an autoradiograph of mRNA hybridized with a mixture of probes complementary to mRNA encoding cyclophilin and β-globin. Hybridized signal-band densities were normalized to corresponding 18S rRNA bands. The bottom panel is an ethidium bromide (EtBr) stained gel before blotting.

FIG. 2 is a Northern blot autoradiograph. The conditions under which the mRNA was isolated and purified, as well as the amounts, are indicated above each lane. The mRNA was hybridized to a probe complementary to cyclophilin mRNA.

FIG. 3 is a series of autoradiographs from agarose gels run at various stages during the generation of a cDNA library. The autoradiograph in panel A was generated from single-stranded cDNA, which was isolated after step 1 of Example 2. The autoradiographs in panels B and C were generated from double-stranded cDNA, which was isolated after step 4 of Example 2. The gel represented in panel B was run under native conditions, while the gel represented in panel C was run under denaturing conditions.

FIG. 4 is a two-paneled autoradiograph from a Southern blot analysis with a probe directed to the 5′-end of cannabinoid receptor-1 (CB1). The top panel corresponds to the conventional cDNA library constructed from total trigeminal ganglion (TG) mRNA, while the bottom panel corresponds to the enriched cDNA library constructed from enriched TG mRNA. Both cDNA libraries were divided into 50 pools. The panels represent 19 of these 50 pools. The pool numbers are indicated above each lane in the autoradiograph. The number of clones per pool is indicated for both the conventional and enriched cDNA.

### DETAILED DESCRIPTION

The disclosed materials, compositions, and methods may be understood more readily by reference to the following detailed description of specific aspects of the materials and methods and the Examples included therein and to the Figures and the previous and following description.

But before the present materials, compositions, methods, articles, and/or devices are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed, and that while specific reference of each various individual and collective combinations and permutation of these compounds may not be explicitly disclosed, each is specifically contemplated and described herein. For example, if a nucleic acid is disclosed and discussed and a number of modifications that can be made to a number of nucleotides in the nucleic acid are discussed, each and every combination and permutation of the nucleic acid and the modifications to the nucleotides that are possible are specifically contemplated unless specifically indicated to the contrary. Thus, if a class of substituents A, B, and C are disclosed as well as a class of substituents D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### DEFINITIONS

In this specification and in the claims that follow, reference will be made to a number of terms that shall be defined to have the following meanings:

As used in the specification and the appended claims, the singular forms “a,” “an” and “the” include plural referents unless the context clearly dictates otherwise. Thus, for example, reference to “a mRNA” includes mixtures of two or more mRNAs; reference to “an expression library” includes mixtures of two or more such expression libraries, reference to “the cDNA” includes mixtures of two or more cDNAs, and the like.

“Optional” or “optionally” means that the subsequently described event or circumstance may or may not occur, and that the description includes instances where the event or circumstance occurs and instances where it does not.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate, including a human.

As used herein, “enriched” refers to an increased number as compared to a control. The control can be, e.g., the total cellular DNA as compared to a selected or enriched portion thereof.

There are a variety of molecules disclosed herein that are nucleic acid based, including, for example, the nucleic acids that encode, for example, secretory proteins and membrane bound proteins, as well as any other proteins, receptors, or channels disclosed herein. Examples of nucleic acids described herein include, but are not limited to, DNA, such as cDNA, and RNA, such as mRNA. The disclosed nucleic acids are made up of, for example, nucleotides, nucleotide analogs, or nucleotide substitutes. Non-limiting examples of these and other molecules are discussed herein. It is understood that, for example, when a vector is expressed in a cell, that the expressed in A will typically be made up of A, C, G, and U.

A “nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more nucleotides linked together. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance, for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA (1989) 86:6553-6556).

Nucleic acids, such as, oligonucleotides to be used as primers and probes described herein can be made using standard chemical synthetic methods or can be produced using enzymatic methods or any other known method. Such methods can range from standard enzymatic digestion followed by nucleotide fragment isolation (see for example, Sambrook et al., Molecular Cloning: A Laboratory Manual, 3d Edition (Cold Spring Harbor Laboratory Press, Cold Spring Harbor, N.Y., 2001) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. (1984) 53:323-356, (phosphotriester and phosphite-triester methods), and Narang et al., Methods Enzymol., (1980) 65:610-620, (phosphotriester method). Protein nucleic acid molecules can be made using known methods such as those described by Nielsen et al., Bioconjug. Chem. (1994) 5:3-7.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH₂ or 0) at the C6 position of purine nucleotides.

Also disclosed herein are compositions including primers and probes, which are capable of interacting with the disclosed nucleic acids, such as the enriched poly A⁺ RNA as disclosed herein. In certain aspects the primers are used to support DNA amplification reactions. Typically, the primers will be capable of being extended in a sequence-specific manner. Extension of a primer in a sequence-specific manner includes any methods wherein the sequence and/or composition of the nucleic acid molecule to which the primer is hybridized or otherwise associated directs or influences the composition or sequence of the product produced by the extension of the primer.

Extension of the primer in a sequence-specific manner therefore includes, but is not limited to, PCR, DNA sequencing, DNA extension, DNA polymerization, RNA translation, transcription, or reverse transcription. Techniques and conditions that amplify the primer in a sequence-specific manner are preferred. In certain aspects the primers are used for the DNA amplification reactions, such as PCR or direct sequencing. It is understood that in certain aspects the primers can also be extended using non-enzymatic techniques, where, for example, the nucleotides or oligonucleotides used to extend the primer are modified such that they will chemically react to extend the primer in a sequence-specific manner. Typically, the disclosed primers hybridize with the disclosed nucleic acids or region of the nucleic acids or they hybridize with the complement of the nucleic acids or complement of a region of the nucleic acids.

The size of the primers or probes for interaction with the nucleic acids in certain aspects can be any size that supports the desired enzymatic manipulation of the primer, such as reverse transcription or the simple hybridization of the probe or primer. A typical primer or probe can be at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

In other aspects, a primer or probe can be less than or equal to 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long.

Methods of Making cDNA Expression Libraries:

Disclosed herein, in one aspect, is a method of making a cDNA expression library enriched for cDNAs that encode secretory or membrane-bound proteins. The disclosed method comprises the steps of (a) isolating membrane-bound polysomal RNA from a selected population of cells; (b) isolating polyadenylated RNA from the isolated membrane-bound polysomal RNA from step (a); and (c) constructing a cDNA expression library from the isolated polyadenylated RNA from step (b). The cDNA expression library prepared by the disclosed method comprises more than about 85%, 90%, 95%, or 99% (or any amount in between) cDNAs that encode secretory or membrane-bound proteins.

Step (a): Isolation of Membrane-Bound Polysomal RNA

The membrane-bound polysomal RNA can be isolated by homogenizing the selected population of cells in a high salt buffer to form a homogenate. The homogenate can be centrifuged to form a supernatant, and the supernatant can be fractionated by centrifugation through a sucrose gradient to isolate the membrane-bound polysomal RNA. Homogenizing a selected population of cells can be performed using methods recognized by one of skill in the art. For example, a Teflon-glass Potter homogenizer or a Dounce homogenizer can be used to homogenize selected cells.

Selected Population of Cells:

The selected population of cells can contain the same type of cells or a mixture of different types of cells. The cells in the selected population of cells can be of any cell type, from any tissue, and from any organism. For example, cells can be derived from any eukaryotic or prokaryotic species and can be differentiated, undifferentiated, de-differentiated, or immortalized. In one aspect, the disclosed methods can be carried out on cells of eukaryotic origin, such as fungus, plant, or animal, or of prokaryotic origin, such as bacteria. A selected population containing cells of eukaryotic origin can be derived from any eukaryotic species, including, but not limited to, mammalian cells (such as rat, mouse, bovine, porcine, sheep, goat, and human), avian cells, fish cells, amphibian cells, reptilian cells, plant cells, yeasts, and the like. In another aspect, the selected population of cells include cells of vertebrates and particularly mammals, more particularly, rats and mice, and more particularly humans. In another aspect, the selected population of cells derived from any of these sources can be primary or can be immortalized cell lines, including, for example hybridomas constructed from different species.

Further, cells can be derived from any tissue in an organism. Examples of useful tissues from which a selected population of cells can be obtained include, but are not limited to, liver, kidney, spleen, bone marrow, thymus, heart, muscle, lung, neural (such as brain, spinal cord, or ganglion), testes, ovary, islet, intestinal, skin, bone, stomach, gall bladder, prostate, bladder, zygotes, embryos, immune cells (including lymphatic), hematopoietic cells, and the like. Examples of plant tissues from which a selected population of cells can be derived include, but are not limited to, leaf tissue, ovary tissue, stamen tissue, pistil tissue, root tissue, gametes, seeds, embryos, and the like.

Some specific examples of the various cell types that can be used to generate the cDNA expression libraries by the methods disclosed herein include, but are not limited to, neurons, muscle cells, pancreatic islet/beta cells, cardiocytes, hepatocytes, glomerulocytes, epithelial cells, T cells, B cells, macrophages, eosinophiles, nucleophiles, stem cells, germ cells (i.e., spermatocytes/spermatozoa and oocytes), fibroblast, and follicular cells. Still further examples of cells that can be present in the selected population of cells include, but are not limited to, sensory neurons, such as dorsal root ganglion neurons and cranial nerve sensory ganglion neurons (e.g., trigeminal ganglion neurons). Such cDNA libraries can be generated from these cells taken from organisms under normal basal conditions, under naturally occurring or induced disease states or following some sort of activation, stimulation or other perturbation of the organism, including, for example, genetic, pharmacologic, surgical, pathogenic, or therapeutic manipulations.

The choice of the cell population and the particular RNA from those cells can be made by one of ordinary skill in the art. The choice will depend on the particular desires and aims of the researcher or clinician. For example, one interested in the function of trans-membrane proteins expressed in sensory neurons of the trigeminal ganglion, could decide to construct an enriched expression cDNA library mainly consisting of cDNAs encoding trans-membrane proteins.

High Salt Buffer:

The selected population of cells can be homogenized in a high salt buffer to form a homogenate. The high salt buffer, in one aspect, comprises one or more of at least one salt, a buffer, and sucrose.

“High salt buffer” means a buffer solution with osmolarity more than 300 mosmol/l in which the total salt concentration is from about 150 mM to about 300 mM, from about 150mM to about 250 mM, from about 150 mM to about 200 mM, or at least about 150 mM.

In one aspect, the high salt buffer contains at least one salt and sucrose. In another aspect, the high salt buffer solution contains more than one salt and sucrose. Suitable salts that can be used in the high salt buffer include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-p-chlorobenzyl−2-pyrrolidin-1′-ylmethylbenzimidazole, diethylamine and other alkylamines, piperazine and tris(hydroxymethyl)aminomethane; alkali metal salts, such as but not limited to lithium, sodium, and potassium; alkali earth metal salts, such as but not limited to barium, calcium and magnesium; transition metal salts, such as but not limited to zinc, aluminum, and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

Specific salts that can be present in the high salt buffer include, but are not limited to, ammonium chloride, lithium chloride, sodium chloride, potassium chloride, magnesium chloride, calcium chloride, zinc chloride, ammonium bromide, lithium bromide, sodium bromide, potassium bromide, magnesium bromide, calcium bromide, zinc bromide, ammonium hydroxide, lithium hydroxide, sodium hydroxide, potassium hydroxide, magnesium hydroxide, calcium hydroxide, zinc hydroxide, ammonium sulfate, lithium sulfate, sodium sulfate, potassium sulfate, magnesium sulfate, calcium sulfate, zinc sulfate, ammonium nitrate, lithium nitrate, sodium nitrate, potassium nitrate, magnesium nitrate, calcium nitrate, and zinc nitrate. For example, the high salt buffer can contain potassium chloride and/or magnesium chloride.

While the total salt concentration of the high salt buffer can be as described above, the concentration of an individual salt in the high salt buffer can be from about 1 mM to about 300 mM salt, from about 100 mM to about 200 mM, or from about 1 mM to about 50 mM. For example, a salt can be present in the high salt buffer at a concentration of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 mM, where any of the stated values can form an upper and/or lower endpoint when appropriate. In one aspect, the high salt buffer can contain about 150 mM of potassium chloride and about 5 mM of magnesium chloride.

The high salt buffer also contains at least one buffering reagent. For example, suitable buffering reagents include Tris, MOPS, HEPES, phosphate, etc. The pH will vary depending upon the particular buffer being used, but generally the pH will be in the range of about 7 to about 7.5. In one aspect, the pH of the high salt buffer is about 7.0, 7.1, 7.2, 7.3, 7.4, or 7.5, where any of the stated values can form an upper and/or lower endpoint when appropriate.

The buffer can be present in the high salt buffer at a concentration that is sufficient to prevent a significant change in pH (i.e., a pH change of more than 1) during the course of the homogenization. For example, the concentration of buffer in the high salt buffer can be from about 1 mM to about 100 mM, from about 25 mM to about 75 mM, or from about 50 mM. For example, a buffer can be present in the high salt buffer at a concentration of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mM, where any of the stated values can form an upper and/or lower endpoint when appropriate.

In a further aspect, the high salt buffer lacks a detergent additive.

In another aspect, the high salt buffer contains a detergent additive. Suitable detergent additives include, but are not limited to, glycerol, polyoxyethylene ethers (“tritons”), such as nonaethylene glycol octylcyclohexyl ether (“TRITON” X-100), polyglycol ethers, particularly polyalkylene alkyl phenyl ethers, such as nonaethylene glycol octylphenyl ether (“NONIDET” P-40 or IGEPAL CA-630), polyoxyethylene sorbitan esters, such as polyoxyethylene sorbitan monolaurate (“TWEEN”-20), polyoxyethylene ethers, such as polyoxyethylene lauryl ether (C₁₂E₂₃) (“BRIJ”-35), stearyl ether (C₁₈E₂₃) (“BRIJ”721), N,N-bis[3-gluconamido-propyl]cholamide (“BIGCHAP”), decanoyl-N-methylglucamide, glucosides such as octylglucoside, 3-[{3-cholamidopropyl}dimethylammonio]−1-propane sulfonate, decylmaltoside, and the like.

In yet another aspect, the high salt buffer contains sucrose. The sucrose can be present at a concentration of from about 0.1 M to about 1.6 M, from about 0.4 to about 1.2 M, or about 0.8 M. For example, sucrose can be present in the high salt buffer at a concentration of about 0.1, 0.2, 0.3, 0.4, 0.5, 0.6, 0.7, 0.8, 0.9, 1.0, 1.1, 1.2, 1.3, 1.4, 1.5, or 1.6 M, where any of the stated values can form an upper and/or lower endpoint when appropriate.

The high salt buffer can further contain additional components, such as preservatives, additives, vanadyl ribonucleased complexes, protease inhibitors, RNAse inhibitors, and the like.

The homogenate formed by homogenizing the selected population of cells in a high salt buffer can be centrifuged to form a supernatant. Centrifugation is well known in the art. In one aspect, the centrifugation can take place in a Sorvall SS-34 rotor. The speed of centrifugation can be at, for example, about 5,000 rpm, 10,000 rpm, or 15,000 rpm. In one aspect, the speed of the centrifugation is at least about 5,000 rpm. The time of centrifugation can be from about 5 minutes to 1 h, from about 10 minutes to about 45 minutes, or about 30 minutes. In one aspect, the time of the centrifugation is at least about 10 minutes, or at least about 15 minutes. After centrifugation, the supernatant can be recovered. The supernatant typically contains membranes, mitochondria, free RNA, free ribosomes, and other proteins.

Sucrose Gradient:

The supernatant can be fractionated by centrifugation through a sucrose gradient to isolate membrane-bound polysomal RNA. In one aspect, the sucrose gradient can be a discontinuous sucrose gradient or a linear sucrose gradient. A discontinuous sucrose gradient can have two or more layers of differing sucrose concentrations. For example, a discontinuous sucrose gradient can contain two, three, four, or five layers of differing sucrose concentrations. In one aspect, the discontinuous sucrose gradient can contain at least three layers of differing sucrose concentrations. A linear sucrose gradient can have one layer wherein the concentration of sucrose linearly or non-linealy varies (e.g., increase or decreases) throughout the layer.

A sucrose layer used in the discontinuous or linear sucrose gradient can contain sucrose and a second salt buffer solution. The second salt buffer solution can contain the types and amounts of salts, buffers, and additives as previously described for the high salt buffer. The second salt buffer solution can be the same as the high salt buffer (i.e., the second salt buffer solution contains the same types and amounts of salts, buffer, and additives as the high salt buffer) or different from the high salt buffer (i.e., the second salt buffer solution contains different types and amounts of salts, buffer, and additives as the high salt buffer).

The amount of sucrose in a discontinuous sucrose layer can be from about 1.0 M to about 3 M. For example, a sucrose layer used in a discontinuous sucrose gradient can have a sucrose concentration of about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 M. In one aspect, in a discontinuous sucrose gradient, one layer contains about 2.5 M sucrose in a second salt buffer solution, another layer contains about 2.0 M sucrose in a second salt buffer solution, and a third layer contains about 1.3 M sucrose in a second salt buffer solution. A suitable second salt buffer solution that can be used for a sucrose layer in a sucrose gradient can contain a buffer like HEPES, and salts like MgCl₂ and KCl.

The sucrose gradient can be prepared in any suitable container, such as a centrifuge tube. In one aspect, the sucrose gradient is in a polyallomer centrifuge tube. The size of the container will depend on the amount and scale of the supernatant and sucrose gradient.

The supernatant can be placed in or on the sucrose gradient and be fractionated by centrifugation. The centrifugation can be an inverted ultra-centrifugation. Inverted ultra-centrifugation is well known in the art. In one aspect, the centrifugation can take place on a SW41Ti rotor. The speed of centrifugation can be at, for example, about 35,000 rpm or about 40,000 rpm. In one aspect, the speed of the centrifugation is at least about 40,000 rpm. The time of centrifugation can be from about 1 h to about 5 h, from about 2 h to about 4 h, or about 3 h. In one aspect, the time of the centrifugation is at least about 3 h. After centrifugation, the membrane-bound polysomal mRNA can be isolated.

Isolated Membrane-Bound Polysomal RNA:

The membrane-bound polysomal RNA can be isolated after centrifugation through the sucrose gradient from a membrane film produced by the centrifugation. For example, the membrane film can be isolated and resuspended in a guanidinium solution. Membrane-bound polysomal RNA can be isolated from the guanidinium solution by extraction with a mixture comprising guanidinium solution, water-saturated phenol, and chloroform. (See Sambrook et al., Molecular Cloning: A Laboratory Manual, 3d Edition (Cold Spring Harbor Laboratory Press, Cold Spring Harbor, N.Y., 2001.) Optionally, the membrane film isolated from the centrifugation can be further fractionated in a linear sucrose gradient. The resulting membrane film from this second centrifugation through a sucrose gradient can be isolated and resuspended in guanidinium solution and the membrane-bound polysomal RNA can be extracted as described before.

The step of isolating membrane-bound polysomal RNA can be conducted at from about −20° C. to about 24° C. In one aspect, the temperature of membrane-bound polysomal RNA isolation can be from about −15° C. to about 24° C., from about −10° C. to about 25° C., from about −5° C. to about 20° C., from about 0° C. to about 15° C., or from about 4° C. to about 10 C. In another aspect, the temperature of membrane-bound polysomal RNA isolation can be about −20, −18, −16, −14, −12, −10, −8, −6, −4, −2, 0, 2, 4, 6, 8, 10, 12, 14, 16, 18, 20, 22, 24° C., where any of the stated values can form an upper and/or lower endpoint when appropriate. In yet another aspect, the temperature of membrane-bound polysomal RNA isolation can be about −19, −17, −15, −13, −11, −9, −7, −5, −3, −1, 3, 5, 7, 9, 11, 13, 15, 17, 19, 21, 23° C., where any of the stated values can form an upper and/or lower endpoint when appropriate. In a further aspect, the temperature of membrane-bound polysomal RNA isolation can be from about 0° C. to about 4° C.

Step (b): Isolation of Polyadenylated RNA

Polyadenylated (poly A⁺) RNA can be isolated from the isolated membrane-bound polysomal mRNA. For example, polyadenylated RNA is isolated by oligo-dT cellulose affinity purification. Suitable oligo-dT cellulose affinity chromatography protocols are described in Chen et al., Nature (1995) 377:428-431 and Akopian et al., Nature (1996) 379:257-262.

The process of isolating polyadenylated RNA can achieve from about 90% to about 98% purification of RNA associated with tight membrane-bound polysomes; that is, the isolated RNA can contain from about 90% to about 98% tight membrane-bound polysomal, polyadenylated RNA. In one aspect, level of purification of membrane-bound polysomal RNA, i.e., the amount of tight membrane-bound polysomal polyadenylated RNA expressed in percentages, can be, for example, about 75, 76, 77, 78, 79, 80, 81, 82, 83, 84, 85, 86, 87, 88, 89, 90, 91, 92, 93, 94, 95, 96, 97, 98, 99, or 100%, where any of the stated values can form and upper and/or lower endpoint when appropriate. In one aspect, the polyadenylated RNA can contain more than 90%, from about 90% to about 98%, or from about 95% to about 98% tight membrane-bound polysomal polyadenylated RNA.

Step (c): Construction of cDNA Expression Library

A cDNA expression library can be constructed from the isolated polyadenylated RNA. Examples of general methods for constructing cDNA expression libraries include Chen et al., Nature (1995) 377:428-431 and Akopian et al., Nature (1996) 379:257-262, which describe suitable methods for generating a cDNA expression library from polyadenylated RNA.

Delivery:

The cDNA expression libraries disclosed herein can be constructed in an appropriate expression vector, such as pcDNA3 and pRK7. Other vectors for delivery into cells are readily available and well known to those skilled in the art. There are a number of other compositions and methods, however, which can also be used to deliver nucleic acids, such as cDNA, into cells. These methods and compositions can largely be broken down into two classes: viral based delivery systems and non-viral based delivery systems. For example, the nucleic acids can be delivered through a number of direct delivery systems such as, biolistic (“gene gun”) technology, electroporation, lipofection, calcium phosphate precipitation, plasmids, viral vectors, viral nucleic acids, phage nucleic acids, phages, cosmids, or via transfer of genetic material in cells or carriers such as cationic liposomes.

Appropriate means for transfection, including viral vectors, chemical transfectants, or physico-mechanical methods such as electroporation and direct diffusion of DNA, are described by, for example, Wolff et al., Science (1990) 247:1465-1468; and Wolff Nature (1991) 352:815-818. Such methods are well known in the art and readily adaptable for use with the compositions and methods described herein. In certain cases, the methods will be modified to specifically function with large DNA molecules. Further, these methods can be utilized to target certain cell populations by using the targeting characteristics of the carrier.

Transfer vectors can be any nucleotide construction used to deliver genes into cells (e.g., a plasmid), or as part of a general strategy to deliver genes, e.g., as part of recombinant retrovirus or adenovirus (Ram et al., Cancer Res. (1993) 53:83-88).

As used herein, plasmid or viral vectors are agents that transport the disclosed nucleic acids, such as cDNA into cells without degradation and include a promoter yielding expression of the gene in cells into which it is delivered. In some aspects the cDNAs are derived from either a virus or a retrovirus. Viral vectors are, for example, Adenovirus, Adeno-associated virus, Herpes virus, Vaccini a virus, Polio virus, AIDS virus, neuronal trophic virus, Sindbis and other RNA viruses, including these viruses with the HIV backbone. Also suitable are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker genes than other viral vectors, and for this reason are commonly used vectors. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector that has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### Retroviral Vectors

A retrovirus is an animal virus belonging to the virus family of Retroviridae, including any types, subfamilies, genus, or tropisms. Retroviral vectors, in general, are described by Venna, I. M., Retroviral vectors for gene transfer in “Microbiology” 1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan Science (1993) 260:926-932, the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. As with other types of vectors, it is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the cDNA of choice is transfected into these cell lines, the vector containing the gene of interest is replicated and packaged into new retroviral particles, by the machinery provided in cis by the helper cell. The genomes for the machinery are not packaged because they lack the necessary signals.

### Adenoviral Vectors

The construction of replication-defective adenoviruses has been described (Berkner et al., J Virology (1987) 61:1213-1220; Massie et al., Mol. Cell. Biol. (1986) 6:2872-2883; Haj-Ahmad et al., J Virology (1986) 57:267-274; Davidson et al., J Virology (1987) 61:1226-1239; Zhang BioTechniques (1993) 15:868-872). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J Clin Invest (1993) 92:1580-1586; Kirshenbaum, J Clin Invest (1993) 92:381-387; Roessler, J Clin Invest (1993) 92:1085-1092; Moullier, Nature Genetics (1993) 4:154-159; La Salle, Science (1993) 259:988-990; Gomez-Foix, J Biol Chem (1992) 267:25129-25134; Rich, Human Gene Therapy (1993) 4:461-476; Zabner, Nature Genetics (1994) 6:75-83; Guzman, Circulation Research (1993) 73:1201-1207; Bout, Human Gene Therapy (1994) 5:3-10; Zabner, Cell (1993) 75:207-216; Caillaud, Eur. J Neuroscience (1993) 5:1287-1291; and Ragot, J Gen Virology (1993) 74:501-507). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology (1970) 40:462-477; Brown and Burlingham, J Virology (1973) 12:386-396; Svensson and Persson, J Virology (1985) 55:442-449; Seth et al., J Virology (1984) 51:650-655; Seth et al., Mol. Cell. Biol. (1984) 4:1528-1533; Varga et al., J Virology (1991) 65:6061-6070; Wickham et al., Cell (1993) 73:309-319).

A viral vector can be one based on an adenovirus which has had the E1 gene removed and these virons are generated in a cell line such as the human 293 cell line. In another aspect both the E1 and E3 genes are removed from the adenovirus genome.

### Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. In another aspect, aspect of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

Inn another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### Large Payload Viral Vectors

Molecular genetic experiments with large human herpesviruses have provided a means whereby large heterologous DNA fragments can be cloned, propagated and established in cells permissive for infection with herpesviruses (Sun et al., Nature Genetics (1994) 8:33-41; Cotter and Robertson, Curr. Opin. Mol. Ther. (1999) 5:633-644). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable. The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

### Non-Nucleic Acid Based Delivery

The disclosed cDNA can be delivered to cells in a variety of ways. For example, the compositions can be delivered through mechanical means, through electroporation, or through lipofection, or through calcium phosphate precipitation. The delivery mechanism chosen will depend in part on the type of cell.

Thus, the compositions can comprise, in addition to the disclosed cDNA or vectors, for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al., Am J Resp Cell Mol Biol (1989) 1:95-100; Felgner et al., Proc Natl Acad Sci USA (1987) 84:7413-7417; U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous cDNA into the cells (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art.

Integration Sequences:

The nucleic acids that are delivered to cells, and which are to be integrated into a cell's genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can become integrated into the host genome.

Other general techniques for integration into a cell's genome include, for example, systems designed to promote homologous recombination with the cell genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

In one aspect, the cDNA can include recognition sites for restriction enzymes, such as EcoRI and XhoI.

Expression Controlling Systems:

The nucleic acids that are delivered to cells typically contain expression-controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### Viral Promoters and Enhancers

Suitable promoters controlling transcription from vectors in mammalian host cells may be obtained from various sources, for example, the genomes of viruses such as: polyoma viruses, Simian Virus 40 (SV40), adenovirus, retroviruses, hepatitis-B virus, and cytomegalovirus, or from heterologous mammalian promoters, e.g., β-actin promoter. The early and late promoters of the SV40 virus are conveniently obtained as an SV40 restriction fragment which also contains the SV40 viral origin of replication (Fiers et al., Nature (1978) 273:113). The immediate early promoter of the human cytomegalovirus is conveniently obtained as a HindIII E restriction fragment Greenway et al., Gene (1982) 18:355-360. Of course, promoters from the host cell or related species also are useful herein.

Enhancer generally refers to a sequence of DNA that functions at no fixed distance from the transcription start site and can be either 5′ (Laimins et al., Proc Natl Acad Sci (1981) 78:993) or 3′ (Lusky et al., Mol Cell Bio (1983) 3:1108) to the transcription unit. Furthermore, enhancers can be within an intron (Banerji et al., Cell (1983) 33:729) as well as within the coding sequence itself (Osborne et al., Mol Cell Bio (1984) 4:1293). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical treatments that trigger their function. Systems can be activated by reagents such as tetracycline and dexamethasone. There are also ways to enhance viral vector gene expression by exposure to irradiation, such as gamma irradiation, or alkylating chemotherapy drugs.

In certain aspects, the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full-length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

### Additional Sequences

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′-untranslated regions also include transcription termination sites. The transcription unit can contain a polyadenylation region. One benefit of this region is that it increases the likelihood that the transcribed unit will be processed and transported like mRNA. The identification and use of polyadenylation signals in expression constructs is well established. Homologous polyadenylation signals can be used in the transgene constructs. In certain transcription units, the polyadenylation region is derived from the SV40 early polyadenylation signal and consists of about 400 bases. It is also suitable that the transcribed units contain other standard sequences alone or in combination with the above sequences improve expression from, or stability of, the construct.

### Marker Products

Expression vectors can include a nucleic acid sequence encoding a marker product. This marker product is used to determine if the gene has been delivered to the cell and once delivered is being expressed. Suitable marker genes include, but are not limited to, the E. Coli lacZ gene, which encodes β-galactosidase, and green fluorescent protein.

In some aspects, the marker may be a selectable marker. Examples of suitable selectable markers for mammalian cells include, but are not limited to, dihydrofolate, reductase (DHFR), thymidine kinase, neomycin, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells.

These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection, which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern and Berg, J Molec Appl Genet (1982) 1:327), mycophenolic acid (Mulligan and Berg, Science (1980) 209:1422), or hygromycin (Sugden et al., Mol Cell Biol (1985) 5:410-413). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

cDNA Expression Libraries:

Disclosed herein are cDNA expression libraries enriched for cDNAs that encode secretory or membrane-bound proteins. In one aspect, the cDNA expression library comprises more than 90% cDNAs that encode secretory and membrane-bound proteins. In another aspect, the cDNA expression libraries prepared according to the disclosed methods can contain more than about 95%, more than about 96%, more than about 97%, or more than about 98% cDNAs that encode secretory and membrane-bound proteins. Still further, the cDNA expression libraries described herein can contain from about 90% to about 98% or from about 95% to about 98% cDNAs that encode secretory and membrane-bound proteins. Such expression libraries can be prepared by the methods disclosed herein.

The cDNA expression libraries described herein can be constructed from any isolated polyadenylated RNA. For example, the polyadenylated RNA used to construct a cDNA expression library can be polyadenylated membrane-bound polysomal RNA, for example, polyadenylated membrane-bound polysomal mRNA. Also, polyadenylated RNA can be isolated from any cell, such as sensory neurons and other cells as previously described. In one aspect, the polyadenylated RNA encodes for secretory and membrane-bound proteins.

Secretory and membrane-bound proteins that can be encoded by the polyadenylated RNA can play important roles in, among other things, the formation, differentiation and maintenance of multicellular organisms. The fate of many individual cells, e.g., proliferation, migration, differentiation, or interaction with other cells, is typically governed by information received from other cells and/or the immediate environment. This information is often transmitted by secreted polypeptides (for instance, mitogenic factors, survival factors, cytotoxic factors, differentiation factors, neuropeptides, and hormones) which are, in turn, received and interpreted by diverse cell receptors or membrane-bound proteins. Such membrane-bound proteins and cell receptors include, but are not limited to, neurotransmitter receptors, cytokine receptors, receptor kinases, ion channels, receptor phosphatases, metabolic/metabotropic enzymes, receptors involved in cell-cell interactions, and cellular adhesion molecules like selectins and integrins. For instance, transduction of signals that regulate cell growth and differentiation is regulated in part by phosphorylation of various cellular proteins. Protein tyrosine kinases, enzymes that catalyze that process, can also act as growth factor receptors. Examples include fibroblast growth factor receptor and nerve growth factor receptor.

Secretory and membrane-bound proteins, such as receptor molecules, have various industrial applications, including as pharmaceutical and diagnostic agents. Receptor immunoadhesins, for instance, can be employed as therapeutic agents to block receptor-ligand interactions. The membrane-bound proteins can also be employed for screening of potential peptide or small molecule activators or inhibitors of the relevant receptor/ligand interaction, which is discussed below.

The cDNAs contained in the cDNA expression libraries described herein contain a high percentage of full-length cDNAs, for example, about 50, 55, 60, 65, 70, 75, 80, 85, 90, 95, or 100% full-length cDNAs, where any of the stated values can form an upper and/or lower endpoint where appropriate. The “full-length cDNA” herein means that the cDNA contains the ATG codon, which is the start point of translation therein. The untranslated regions upstream and downstream of the protein-coding region, both of which are naturally contained in natural mRNAs, are not indispensable. In one aspect, the full-length cDNAs contain the stop codon.

Expression Cloning:

When cDNA is incorporated into an expression vector, as previously described, the resulting cDNA expression library can be divided into a number of pools (M), which contain a number of independent clones (N). Each pool can be transfected into the specialized cell type for which an appropriate functional assay can be performed. Upon completion of a functional assay, the positive pool can be divided into smaller sub-pools. These identified sub-pools can then be transfected into a specialized cell type and a second functional assay can be performed. The steps of transfection, expression, functional analysis, and identification of positive sub-pools can be repeated again and again until a single, functionally positive cDNA clone is identified.

In general, the sensitivity of the expression cloning is directly related to the sensitivity of the functional assay used and the size (N×M) of the cDNA expression library. High sensitivity functional assays are capable of detecting signal from the gene of interest when N is greater than about 1500; moderate sensitivity functional assays are capable of detecting signal from the gene of interest when N is from about 500 to 1500. And finally, low sensitivity functional assays are capable of detecting signal from the gene of interest when N is less than about 500. Some popular and commonly used functional assays, such as the agonist stimulated GTPγS binding assay, the radioreceptor binding assay, the cAMP-accumulation assay, etc., are not, however, able to register signal when N is greater than about 300-400.

The sensitivity of the functional assay depends on the nature of the functional assay itself and the amount of the protein of interest which may be translated from one of the cDNA clones that is present in the pool of N clones. There are theoretical limits to the extent to which functional assay sensitivity may be enhanced. However, relatively large amounts of the protein of interest can be generated when efficient transfection is achieved and powerful expression vectors are employed.

The total number of cDNAs in a representative cDNA library can be reflected as N×M. For a conventional cDNA library, M×N can be at least about 150,000-200,000. If a functional assay is presumed to be relatively insensitive, then for it to be successful, N can be no more than about 300-400; thus, M will be about 500. Such a high value for M can make the expression cloning approach excessively bulky, tedious, time-consuming, cumbersome, and ineffective. Thus, it may be desirable to reduce M for the particular expression cloning effort. A reduction in M can be accomplished in at least two ways:

First, an “ideal” cDNA library, which contains only full-length clones, can be constructed. Because only full-length cDNA can be faithfully translated, excessive non-full length clones can reduce the library's sensitivity. A “quality cDNA library,” as taught herein, typically has no more than about 80% non-full-length cDNA clones. Preferably, the cDNA expression library described herein has more than about 20% full-length clones.

Second, in most cell types, including neurons, the membrane-bound polysomes represent only from about 5 to 20% of the total cell ribosomal population. Moreover, only from about 5 to 15% of genes are translated on membrane-bound polysomes (Mechler, Methods in Enzymol (1987) 152:241-248). Therefore, the construction of a cDNA expression library from mRNA translated on membrane-bound polysomes allows for the effective decrease (5-10 times) in the value of M, and thereby increases the sensitivity of the expression cloning approach. Such an enriched cDNA expression library allows for the functional screening of 80-120 pools of 300-400 clones to identify the gene of interest.

Methods for Using cDNA Expression Libraries:

The described cDNA expression libraries can have wide applications and many uses. For example, a DNA microarray comprising the cDNAs of the expression library can be used to identify mRNA that encodes specific proteins of interest. Furthermore, a microarray containing mainly trans-membrane proteins expressed in tissue/cell types of interest can be prepared from the disclosed cDNA expression libraries. Further, because sites of action for many synthetic drugs are unknown, the libraries generated using the method disclosed herein can be used for high-throughput screening of potential drugs by applying compounds to the library or to proteins expressed by the cDNAs of the library. Specifically, for example, mechanisms of chemical (e.g., bradykinins, histamines, cytokines, and sphingolipids) and mechanical activation of nociceptors (pain-sensing neurons) are unknown; thus, the disclosed cDNA expression libraries can be used to identify the involved receptors/channels.

Screening:

In one aspect, disclosed herein is a method of screening for selected membrane-bound proteins or secretory proteins. This method comprises the steps of (a) contacting the proteins expressed by a cDNA expression library disclosed herein with a marker that binds the selected membrane-bound proteins or secretory proteins and (b) detecting the bound marker, the bound marker indicating the presence of the selected membrane-bound proteins or secretory proteins.

In general, expression proteins can be blotted onto a filter and contacted with the marker. The marker can be any compound, such as an antibody or ligand, that will bind to an expression protein of interest and is detectable by methods described below.

Methods for detecting the bound marker include, but are not limited to, enzyme-linked immunosorbent assay (ELISA), immuno-PCR, immunocytochemistry, ligand binding, radioimmunoassay, immunoprecipitation, and immunoblotting. In one aspect, suitable methods of direct/indirect detection of the markers can be calorimetric, fluorometric, chemiluminescent, or spectroscopic, including radiometric.

In another aspect, disclosed herein is a method of screening for cDNAs that encode selected membrane-bound proteins. This method comprises the steps of (a) contacting the cDNA expression library disclosed herein with a nucleic acid that selectively hybridizes under stringent condition with cDNA that encodes selected membrane-bound proteins and (b) detecting the hybridizing cDNA, the hybridizing cDNA indicating the presence of the cDNAs that encode the selected membrane-bound proteins.

Parameters for selective hybridization between two nucleic acid molecules are well known to those of skill in the art. For example, in some aspects, selective hybridization conditions can be defined as stringent hybridization conditions. For example, stringency of hybridization is controlled by both temperature and salt concentration of either or both of the hybridization and washing steps and depends upon the length of the probe over which it exhibits complementarity to its target. Nucleic acid duplex or hybrid stability is expressed as the melting temperature or Tm, which is the temperature at which a nucleic acid probe dissociates from a target nucleic acid. This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE). Assuming that a 1% mismatch results in a 1° C. decrease in the Tm, the temperature of the final wash in the hybridization reaction is reduced accordingly (for example, if sequence having >95% identity with the probe are sought, the final wash temperature is decreased by 5° C.). In practice, the change in Tm can be between 0.5° C. and 1.5° C. per 1% mismatch. Stringent conditions involve hybridizing at 68° C. in 5×SSC/5×Denhardt's solution/1.0% SDS, and washing in 0.2×SSC/0.1% SDS at room temperature. Moderately stringent conditions include washing in 3×SSC at 42° C. for oligonucleotide probes of approximately 40-50 bp. The parameters of salt concentration and temperature can be varied to achieve the optimal level of identity between the probe and the target nucleic acid. Additional guidance regarding such conditions is readily available in the art, for example, in Sambrook, et al., Molecular Cloning, A Laboratory Manual, 3d Edition Cold Spring Harbor Press, New York, N.Y. (2001); and Ausubel et al., eds., Current Protocols in Molecular Biology, John Wiley & Sons, New York, N.Y., at Unit 2.10 (1995).

Hybridization temperatures are typically higher for DNA-RNA and RNA-RNA hybridizations. A preferable stringent hybridization condition for a DNA:DNA hybridization can be at about 68° C. (in aqueous solution) in 5×SSC or 5×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some aspects, selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of the limiting nucleic acid is bound to the non-limiting nucleic acid. Typically, the non-limiting primer is in, for example, 10- or 100- or 1000-fold excess. This type of assay can be performed under conditions where both the limiting and non-limiting primer are for, example, 10-fold or 100-fold or 1000-fold below their Kd value, or where only one of the nucleic acid molecules is 10-fold or 100-fold or 1000-fold or where one or both nucleic acid molecules are above their Kd value.

Another way to define selective hybridization is by looking at the percentage of primer that gets enzymatically manipulated under conditions where hybridization is required to promote the desired enzymatic manipulation. For example, in some aspects selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 percent of the primer molecules are extended. Suitable conditions also include those suggested by the manufacturer or indicated in the art as being appropriate for the enzyme performing the manipulation.

Methods for detecting the hybridization of nucleic acids to cDNA include, but are not limited to, in situ hybridization and Northern or dot/slot blotting (with oligonucleotide or ribonucleic acid probes) or RT-PCR. In one aspect, suitable methods of direct/indirect detection of the nucleic acids that selectively hybridize to cDNA can be calorimetric, fluorometric, chemiluminescent, or spectroscopic, including radiometric.

The secretory or membrane-bound proteins (i.e., receptors, channels, enzymes, and the like) identified by methods of using the cDNA expression libraries disclosed herein can be used as targets for screening agents that modulate these secretory or membrane-bound proteins in a desired way. One method of screening for agents that modulate selected membrane-bound proteins or secretory proteins comprises the steps of (a) contacting an agent to be screened with membrane bound proteins or secretory proteins selected by the methods disclosed herein and (b) detecting an increase or decrease in a selected function of the membrane-bound or secretory proteins as compared to the selected membrane-bound proteins or secretory proteins in the absence of the agent, an increase or decrease in function indicating an agent that modulates the selected membrane-bound or secretory proteins. Also disclosed are agents identified through this method.

A selected function of the membrane-bound or secretory proteins can be specific binding to an agonist, antagonist, modulator, or a co-factor. Also, in a cellular environment, a selected function can be intracellular signaling. In one aspect, the selected function of the membrane-bound or secretory proteins can be specific activity. The specific activity can be channel activity (e.g., ion flux measured by FLIPR), metabotropic activity (e.g., G-protein activation measured by GTPγS binding), or enzyme activity (e.g., metabolic conversion measured by modification of substrate or accumulation of product).

The contacting step can comprise contacting a cell that expresses the membrane bound proteins or secretory proteins. Alternatively, the contacting step can involve contacting the agent to proteins attached to columns or other platforms.

In a still further aspect, disclosed herein is a method of screening for agents that modulate expression of selected membrane-bound proteins or secretory proteins. This method comprises the steps of (a) contacting an agent to be screened with a test cell that expresses the selected membrane-bound or secretory proteins encoded by the cDNAs identified by the methods disclosed herein and (b) detecting an increase or decrease in expression of the membrane-bound or secretory proteins as compared to the selected membrane-bound proteins or secretory proteins in the absence of the agent, an increase or decrease in expression indicating an agent that modulates expression of the selected membrane-bound or secretory proteins.

In another aspect, disclosed herein is a method of screening for agents that modulate expression of selected membrane-bound proteins or secretory proteins. The method comprises the steps of (a) contacting an agent to be screened with a test cell that expresses the selected membrane-bound or secretory proteins identified by the methods disclosed herein and (b) detecting an increase or decrease in expression of the membrane-bound or secretory proteins in the test cell as compared to expression of the selected membrane-bound proteins or secretory proteins in a control cell in the absence of the agent, an increase or decrease in expression in the test cell indicating an agent that modulates expression of the selected membrane-bound or secretory proteins.

In yet another aspect, disclosed herein is a method of screening for agents that modulate expression of selected membrane-bound proteins or secretory proteins. This method comprises the steps of (a) contacting an agent to be screened with a test cell that expresses the selected membrane-bound or secretory proteins and (b) comparing nucleic acid expression by the cell with the cDNAs identified by the methods disclosed herein, an increase or decrease in expression by the test cell as compared to the expression by a control cell in the absence of the agent indicating an agent that modulates expression of the selected membrane-bound or secretory protein. Comparing nucleic acid expression by the cell with the cDNAs can include comparing cellular mRNA to corresponding cDNAs.

The proteins, receptors, channels, enzymes, and the like identified by the cDNA expression libraries disclosed herein can be used as targets for any combinatorial technique to identify molecules or macromolecular molecules that interact with these disclosed compositions in a desired way. Also disclosed are the compositions that are identified through combinatorial techniques or screening techniques in which the disclosed proteins, receptors, channels, enzymes and the like are used as the target in a combinatorial or screening protocol.

It is understood that when using the disclosed targets in combinatorial techniques or screening methods, molecules, such as macromolecular molecules, will be identified that have particular desired properties, such as inhibition or activation or the target molecule's function. The molecules identified and isolated when using the disclosed compositions are also disclosed.

It is understood that the disclosed methods for identifying molecules that inhibit the interactions between these molecules and a target identified by expression cloning with the disclosed enriched cDNA expression libraries or the proteins encoded by the cDNA of the expression libraries can be performed using high-throughput means. For example, putative inhibitors can be identified using Fluorescence Resonance Energy Transfer (FRET) to quickly identify interactions. The underlying theory of the techniques is that when two molecules are close in space, i.e., interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decreasing or increasing signal can be correlated to the presence or absence of the putative inhibitor. Any signaling means can be used. For example, disclosed are methods of identifying an inhibitor of the interaction between any two of the disclosed molecules comprising the steps of (a) contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor and (b) measuring Fluorescence Resonance Energy Transfer (FRET), in the presence of the putative inhibitor and in the absence of the putative inhibitor, wherein a decrease in FRET in the presence of the putative inhibitor as compared to FRET measurement in its absence indicates the putative inhibitor inhibits binding between the two molecules. This type of method can be performed with a cell system as well.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides using the methods taught in Szostak, TIBS (1992) 19:89. In one aspect, a large pool of molecules bearing random and defined sequences is synthesized and subjected to, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, it has been estimated that 1 in 10¹⁰ RNA molecules fold in such a way as to bind small molecule dyes. Techniques aimed at similar goals exist for small organic molecules, proteins, antibodies and other macromolecules known to those of skill in the art. Screening sets of molecules for a desired activity, whether based on small organic libraries, oligonucleotides, or antibodies, is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See e.g., U.S. Pat. Nos. 6,031,071; 5,824,520; 5,596,079; and 5,565,332 which are herein incorporated by reference, at least for their material related to phage display and methods relate to combinatorial chemistry).

One method for isolating proteins that have a given function is described by Roberts and Szostal, Natl Acad Sci USA (1997) 94:12997-13002. This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA, to be translated. In addition, because of the attachment of the puromycin, a peptdyl acceptor that cannot be extended, the growing peptide chain is attached to the puromycin, which is attached to the RNA. Thus, the protein molecule is attached to the genetic material that encodes it. Normal in vitro selection procedures can now be done to isolate functional peptides. Once the selection procedure for peptide unction is complete, traditional nucleic acid manipulation procedures are performed to amplify the nucleic acid that codes for the selected functional peptides. After amplification of the genetic material, new RNA is transcribed with puromycin at the 3′-end, new peptide is translated, and another functional round of selection is performed. Thus, protein selection can be performed in an iterative maimer just like nucleic acid selection techniques. The peptide that is translated is controlled by the sequence of the RNA attached to the puromycin. This sequence can be anything, such as a random sequence engineered for optimum translation (i.e., no stop codons etc.) or a degenerate sequence of a known RNA molecule to look for improved or altered function of a known peptide. The conditions for nucleic acid amplification and in vitro translation are well known to those of ordinary skill in the art and can be performed as in Roberts and Szostak, Proc Natl Acad Sci USA (1997) 94:12997-13002.

Another method for combinatorial methods designed to isolate peptides is described in Cohen et al. (Cohen et al., Proc Natl Acad Sci USA (1998) 95:14272-14277). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature (1989) 340:245-246). Cohen et al. modified this technology so that novel interactions between synthetic or engineered peptide sequences could be identified which bind a molecule of choice. The benefit of this type of technology is that the selection is done in an intracellular environment. The method utilizes a library of peptide molecules that are attached to an acidic activation domain. A peptide of choice is attached to a DNA binding domain of a transcriptional activation protein, such as Gal 4. By performing the two-hybrid technique on this type of system, molecules that bind the peptide of choice can be identified.

Using methodology well known to those of skill in the art, in combination with various combinatorial libraries, one can isolate and characterize those small molecules or macromolecules that bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules that bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin−2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene−2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein, combinatorial methods and libraries include traditional screening methods and libraries as well as methods and libraries used in interative processes.

Microarrays:

Disclosed herein are microarrays comprising cDNAs from the expression libraries disclosed herein or proteins encoded by the cDNA of the expression libraries. DNA microarrays, also known as chips, can have at least one address, being the sequences or part of the sequences set forth in any of the nucleic acid sequences disclosed herein. Also disclosed are chips where at least one address is the sequences or portion of sequences set forth in any of the peptide sequences disclosed herein. Also disclosed are chips wherein at least one address is a variant of the sequences or part of the sequences set forth in any of the nucleic acid sequences disclosed herein. Also disclosed are chips where at least one address is a variant of the sequences or portion of sequences set forth in any of the peptide sequences disclosed herein.

In a further aspect, a microarray of nucleic acids (e.g., cellular mRNA) can be screened by contacting the nucleic acids of the microarray with the cDNAs of the cDNA expression libraries disclosed herein.

Computer-Readable Mediums:

It is understood that the disclosed nucleic acids and proteins can be represented as sequences consisting of the nucleotides or amino acids, respectively. There are a variety of ways to display these sequences, for example the nucleotide guanosine can be represented by G or g. Likewise the amino acid valine can be represented by Val or V. Those of skill in the art understand how to display and express any nucleic acid or protein sequence in any of the variety of ways that exist, each of which is considered herein disclosed. Specifically contemplated herein is the display of these sequences on computer-readable mediums, such as commercially available floppy disks, tapes, chips, hard drives, compact disks, and video disks, or other computer readable mediums. Also disclosed are the binary code representations of the disclosed sequences. Those of skill in the art would understand suitable computer-readable mediums on which the disclosed nucleic acids or protein sequences can be recorded, stored, or saved.

Disclosed are computer-readable mediums comprising the sequences and information regarding the sequences set forth herein. Also disclosed are computer-readable mediums comprising the sequences and information regarding the sequences set forth herein.

Computer-Assisted Drug Design:

The disclosed compositions can be used as targets for any molecular modeling technique to identify either the structure of the disclosed compositions or to identify potential or actual molecules, such as small molecules, which interact in a desired way with the disclosed compositions. The nucleic acids, peptides, and related molecules disclosed herein can be used as targets in any molecular modeling program or approach.

It is understood that when using the disclosed compositions in modeling techniques, molecules, such as macromolecular molecules, will be identified that have particular desired properties, such as inhibition or activation of the target molecule's function. The molecules identified and isolated when using the disclosed compositions are also disclosed. Thus, the products produced using the molecular modeling approaches that involve the disclosed compositions are also considered herein disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to optimize binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass., or SYBYL from Tripos Corp. Madison, Wis. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles, such as Rotivinen et al., Acta Pharmaceutica Fennica (1988) 97:159-166; Ripka, New Scientist (1988) 54-57; McKinaly and Rossmann, Annu Rev Pharmacol Toxiciol (1989) 29:111-122; Perry and Davies, “QSAR: Quantitative Structure-Activity Relationships in Drug Design,” Alan R. Liss, Inc., pp. 189-193, 1989; Lewis and Dean, Proc R Soc Lond (1989) 236:125-162; and, with respect to a model enzyme for nucleic acid components, Askew et al., J Am Chem Soc (1989) 111:1082-1090, review computer modeling of drugs interacting with specific proteins. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

Kits:

Disclosed herein are kits that are drawn to reagents that can be used in practicing the methods disclosed herein. The kits can include any reagent or combination of reagents discussed herein or that would be understood to be required or beneficial in the practice of the disclosed methods. For example, the kits could include primers to perform the membrane-bound polysomal mRNA isolations discussed in certain aspects of the methods, as well as the buffers and enzymes required to use the primers as intended. For example, disclosed is a kit for isolating membrane-bound polysomal mRNA, comprising the salt buffer solution and sucrose gradients described above. Also disclosed are, for example, kits comprising an enriched cDNA expression library and reagents for expression cloning.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

Isolation and Purification of RNA from Membrane-Bound Polysomes:

“Type of RNA” is RNA with a particular name (for example, ATP-gated ion-channel mRNA). “Amount of RNA” is a percentage of the particular RNA compared to the total RNA pool isolated from a defined cell type. For example, small diameter sensory neurons contain 0.01% of PN1 voltage-gated sodium channel mRNA; in other words, 0.1 ng of the PN1 mRNA will be isolated from 1 μg of the total mRNA pool isolated from small diameter sensory neurons. 30-50% of the types of total neuronal mRNA are translated on the membrane-bound polysomes. However, only 5-10% of the amounts of total neuronal mRNA are translated on the membrane-bound polysomes, because amounts of mRNA translated on the free ribosomes are extremely abundant. Thus, the amounts of the β-globin and cyclophilin mRNAs are estimated to be about 0.5-1%, while the amount of the ATP-gated ion channel mRNA is only 0.01%. Generally, receptor and channel mRNAs are moderately or rarely represented. Moreover, some evidence indicates that, although certain mRNA molecules are presented in the cytoplasm, they are not translated at all or are translated at lower rates than other mRNAs in the neuron.

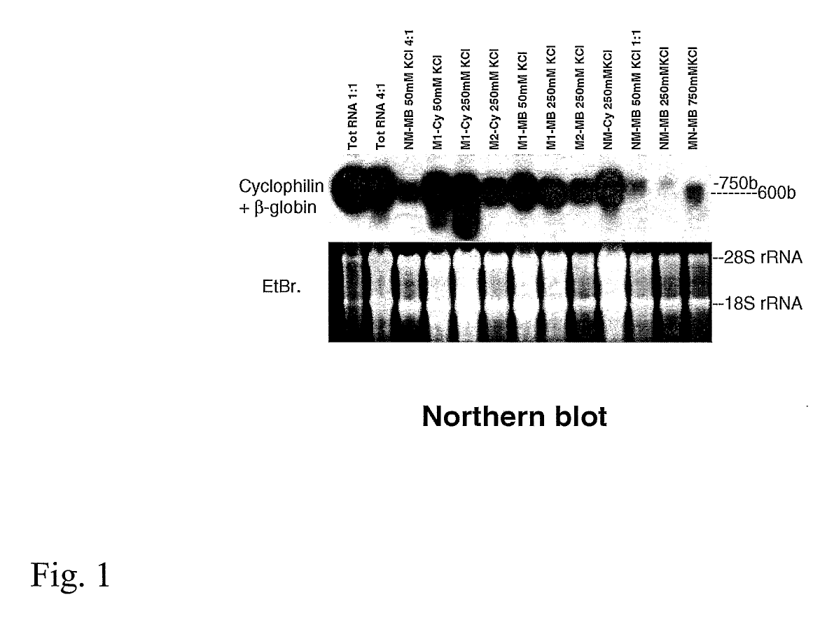

To achieve a sufficient enrichment of receptor and channel cDNA clones in a cDNA expression library, about a 95-99% purification level of the membrane-bound polysomes should be attained. Also, the membrane-bound polysome fraction should be purified from free ribosomes as well as from the ribosome-unbound RNA (i.e., inactive) fraction. The following examples compare two major methods (marked M1 and M2) for purification of the membrane-bound polysomal RNA and compare them with the method disclosed herein (see FIG. 1).

### Comparative Example A

The M1 method was originally described by Ramsey and Steele (Ramsey and Steele, J Neurochem 7 (1977) 28(3):517-527) and is perhaps the most often cited method of isolating membrane-bound polysomes. Since then, many versions of the method have been published (see Ramsey and Steele, Anal Biochem (1979) 92(2):305-313; Rademacher and Steele, J Neurochem (1986) 47(3):953-957).

The M1 method was replicated as it was originally described by Ramsey and Steele (Ramsey and Steele, J Neurochem (1977) 28(3):517-527). That is, adult rat cerebellum tissue was thoroughly homogenized in buffer containing 250 mM sucrose, 250 mM or 50 mM KCl, and no-detergent. This step was done as fast as possible (5-20 minutes) to avoid degradation of RNA. Next, nuclear and microsome fractions were co-precipitated by low speed centrifugation. Supernatant containing free ribosomes and ribosome-unbound RNAs was collected, and RNA was extracted as described in Chomczynski and Sacchi, Anal Biochem (1987) 162(1):156-159. Isolated RNA at this stage was marked M1-Cy (see FIG. 1).

Pellet was homogenized in the presence of 0.1% Triton X−100 to separate the nuclear fraction from the microsomal fraction. The nuclear fraction was removed by low-speed centrifugation. Then, the supernatant containing the microsomal fraction was treated with deoxycholate to strip membrane-bound polysomes from the microsomes. Microsomes (without ribosomes) were removed by an average-speed centrifugation, and supernatant containing the membrane-bound polysomes was collected.

The supernatant was layered onto a discontinuous sucrose gradient (see Ramsey and Steele, J Neurochem (1977) 28(3):517-527) and spun at high speed to precipitate membrane-bound polysomes. RNA was extracted from polysomal pellet as described in Chomczynski and Sacchi, Anal Biochem (1987) 162(1):156-159. Isolated RNA at this stage was marked M1-MB (see FIG. 1).

### Comparative Example B

The M2 method, which is similar to the M1, was described in Sajdel-Sulkowska et. al., J Neurochem (1983) 40(3):670-680. This method was replicated by first thoroughly homogenizing adult rat cerebellum tissue in buffer containing 250 mM sucrose, 250 mM KCl, and no detergent. Nuclear and microsome fractions were co-precipitated by low-speed centrifugation. The supernatant containing free ribosomes and ribosome-unbound RNAs was collected, and RNA was extracted as described in Chomczynski and Sacchi, Anal Biochem (1987) 162(1):156-159. Isolated RNA at this stage was marked M2-Cy (see FIG. 1).

Pellet was homogenized in the presence of both 0.1% Triton X−100 and deoxycholate to separate the membrane bound polysomes from nuclear and microsomal fractions. The nuclear and striped microsomal fractions were removed by an average-speed centrifugation. RNA was extracted from the supernatant fraction, which contained membrane-bound polysomes, as described in Chomczynski and Sacchi, Anal Biochem (1987) 162(1):156-159). Isolated RNA at this stage was marked M2-MB (see FIG. 1).

### Example 1

All of the steps in this example were performed on ice or at 4° C. All stock solutions were autoclaved before use and all sucrose containing buffers were treated with 0.1% DEPC at 70° C. for 3 h. Buffer A was a 50 ml solution containing 150 mM KCl (3.75 ml of 2 M KCl), 5 mM MgCl₂ (0.25 ml of 1 M MgCl₂), 50 mM HEPES pH 7.4 (2.5 ml of 1 M HEPES pH 7.4). Buffer A contained high concentrations of salt to prevent membrane contamination from absorbed free ribosomes. Buffer B was a 50 ml solution containing Buffer A plus 0.8 M sucrose (13.692 g), 20 mM vanadyl ribonuclease complexes, VRC, (Sigma; Ronkonkoma, N.Y.), 30μ/ml of the buffer protease inhibitors P−8340 (Sigma; Ronkonkoma, N.Y.), and 100 U/ml of RNAse inhibitor.

First, 0.5-0.7 g of adult rat trigeminal ganglion tissue (e.g., 30-40 pairs of trigeminal ganglia) was isolated. The tissue was rinsed in ice-cold Buffer A. Next, the tissue was homogenized in 8-12 ml of Buffer B using a Teflon-glass Potter homogenizer. The homogenate was then passed through a 19½ gauge needle and then a 22½ gauge needle using a 10-30 ml-syringe. Next, to sediment nuclei, the homogenate was spun at 10,000 rpm for 15 minutes at 4° C. in a Sorvall SS-34 rotor. The speed of the centrifuge was high because of the presence of 0.8 M sucrose in Buffer B. The supernatant contained much membrane and about 5-20% mitochondria, free RNA, free-ribosome and other proteins. The supernatant, which was a milky suspension, was recovered.

To the supernatant (4 ml) was added 12.5 ml of 2.5 M sucrose in Buffer C. The mixture was gently mixed. The final concentration of sucrose in the mixture was 2.1-2.15 M. In polyallomer centrifuge tubes (12 ml capacity), the mixture (8-8.25 ml) was over-layered by 2.5 ml of 2 M sucrose in Buffer C, then 1 ml of 1.3 M sucrose in Buffer C and 100 U/ml of RNAse inhibitor to therefore provide a discontinuous gradient.

The 2.5 M sucrose in Buffer C solution (50 ml) contained 42.75 g of sucrose, 2.5 ml of 1 M HEPES pH 7.4, 0.25 ml of 1 M MgCl₂, and 3.75 ml of 2 M KCl. The 2.0 M sucrose in Buffer C solution (50 ml) contained 34.23 g sucrose, 2.5 ml of 1 M HEPES (pH=7.4), 0.25 ml of 1M MgCl₂, and 3.75 ml of 2 M KCl. The 1.3 M sucrose in Buffer C solution (50 ml) contained 22.23 g sucrose, 2.5 ml of 1 M HEPES (pH=7.4), 0.25 ml of 1M MgCl₂, and 3.75 ml of 2 M KCl.

The discontinuous gradient was centrifuged for 3 h at 4° C. in a SW41Ti rotor at 40,000 rpm. Membrane appeared as a yellow-green, soft film floating between the 2 M and 1.3 M sucrose layers. Remains of mitochondria, free RNA (rather RNPs), free-ribosomes, uncoupled membrane-bound ribosomes, and other cytoplasmic proteins could be found as sediment and/or stayed in the mix layer (2.15 M sucrose). This membrane film and surrounding solution were collected by Pasteur pipette and resuspended in 3-6 ml of guanidinium solution (50 g guanidinium isotiocianata and 0.5% sorcasin, both from Sigma (Ronkonkoma, N.Y.), 3.52 ml Na-citrate pH 7.0, and 64 ml of water). RNAs were isolated using the guanidinium-acid/phenol/chloroform method. (See Sambrook et al., Molecular Cloning: A Laboratory Manual, 3d Edition (Cold Spring Harbor Laboratory Press, Cold Spring Harbor, N.Y., 2001.) Isolated cytoplasmic RNA was labeled NM-Cy, and RNA isolated from membrane-bound polysome was labeled NM-MB (see FIG. 1.)

Uncoupled membrane-bound polysomes could be further fractionated in a linear (15-45%) sucrose gradient to obtain a polysome-enriched fraction. However, the described method yielded no free RNA or free polysomes and very small amounts of ribosome and disome. It is of note that only polysomes, not ribosomes and disomes, are directly involved in the synthesis of proteins.

Lastly, a two-step affinity purification of poly A⁺ RNA was performed as described in Chen et al., Nature (1995) 377: 428-431 and Akopian et al., Nature (1996) 379:257-262.

Yields of membrane-bound polysomal RNA depend on ionic concentrations of the homogenizing buffers (Ramsey and Steele, Biochem J (1977) 168(1):1-8); therefore, several isolations were performed at different ionic concentrations, which are indicated in FIG. 1 (e.g., NM-MB 50 mM KCl, NM-MB 250 mM KCl, NM-MB 750 mM KCl). Northern blot analysis (FIG. 1) included total RNA (labeled tot RNA 4:1) and a four-times diluted version of total RNA (labeled tot RNA 1:1) isolated as described in Chomczynski and Sacchi, Anal Biochem (1987) 162(1):156-159.

Results:

Since one aim of the membrane-bound polysome fraction purification was to maximally (95-99%) eliminate the presence of ribosome-unbound and free ribosome bound mRNAs (especially abundant ones), the Northern blot was hybridized with a mixed probe against the super-abundant, house-keeping mRNAs cyclophilin and β-globin. Hybridized signal-band densities were normalized against 18S rRNA (see FIG. 1; EtBr-ethidium bromide-stained gel before blotting). It was estimated that the M2 methods gave 70-80% of a purification level for the membrane-bound polysomal mRNA (compare tot RNA 4:1 and M2-MB 250 nM KCl; FIG. 1). The M1 methods gave only 50-60% of a purification level (compare tot RNA 4:1 and M1-MB 250 mM KCl; FIG. 1). The method disclosed in Example 1, however, gave 95-97% of a purification level (compare total RNA 1:1 and NM-MB 250 mM KCl; FIG. 1).

There is a large difference between the 97% observed with the method disclosed in Example 1 and the 80% observed with the next best method, M2. That is, the 97% observed with the method of Example 1 means a 30-fold purification, whereas the 80% observed with the M2 method means only a 5-fold purification. Also, the method of purification indicated by NM 250 mM KCl almost completely eliminated the f-globin mRNA (approximate size is 600 b) from the membrane-bound polysomal mRNAs.

While not wishing to be bound by theory, it is believed that there are two main reasons the disclosed method allows such improved purification levels as compared to the traditional (M1 and M2) methods. First, the homogenization buffer used in methods M1 and M2 is essentially free of any type of detergent. Therefore, there is a substantial percentage (5-20%) of undisrupted (by homogenization) cells that will contaminate the nuclear-microsomal fraction by co-procipitation. Then, these cells are partially or completely disrupted/lysed during pellet homogenization and/or separation of nuclear and microsomal fractions of the M1 and M2 methods. Thus, free ribosomal and ribosome-unbound mRNAs are co-isolated with membrane-bound polysomal RNAs. The disclosed method allows the users to overcome such a problem, because mRNA can be extracted directly from microsomes floating on the surface after an inverted ultra-centrifugation, while the traditional methods strip membrane-bound ribosomes from microsomes and co-precipitate them with any contamination (i.e., free ribosomes of initially undisrupted cells).

Still, while not wishing to be bound by theory, the second reason the disclosed method allows such improved purification levels as compared to the traditional (M1 and M2) methods is believed to be because free ribosomal and, possibly, ribosome-unbound mRNAs can be non-specifically absorbed to microsomes and/or nuclei during isolation procedures. Perhaps, therefore, some authors suggested an existence of two different membrane-bound polysomal fractions—“loose” and “tight” (Cardelli and Pitot, Biochemistry (1977) 16(23):5127-5134; Ramsey and Steele, Biochem J (1977) 168(1):1-8). The inverted ultra-centrifugation of the disclosed method resists absorption to the microsome fraction, because only “tight” membrane-bound ribosomes can remain bound to microsomes.

cDNA Expression Library Construction from Enriched Poly A⁺ RNA:

### Example 2

First, 10 μl of 5× Reverse Transcription Buffer from Invitrogen (Carlsbad, Calif.), 6 μl of 0.1 M DTT from Invitrogen (Carlsbad, Calif.), 2 μl of Xho-oligo(dT)-primer-linker from Stratagene (La Jolla, Calif.), 3 μl of methyl-nucleotide mix (10 mM each of dATP, dTTP, and dGTP, and 5 mM methylated dCTP), and 18 μl of sterile water were mixed together at room temperature. Then, to the mixture was added 9 μl (1-2 μg) of enriched poly A⁺ RNA from Example 1, which was previously heated at 70° C. for 2 minutes and then quickly cooled on ice.

Second, 2.5 μl of Super Script II (200 U/μl) from Invitrogen (Carlsbad, Calif.) was added to the mixture followed by more mixing. In order to estimate the average and maximal sizes of the first strands of cDNAs, a “tracing reaction” was prepared by transferring 3 μl from the main mixture to 0.5 μl (5 μCi) of [α-³²P] dATP (>3000 Ci/mmol). The main (non-radioactive) and “tracing” (radioactive) reaction mixtures were then both incubated at 42° C. for 1 h. The “tracing reaction” was frozen for further analysis on a denaturing agarose gel. The analysis showed that the first strands of cDNA were of high quality with a maximal size of about 13 kilobases (kb) and an average size of about 2.5 kb.

Third, the main reaction mixture was charged at room temperature with 40 μl of 10× Second Strand Buffer from Invitrogen (Carlsbad, Calif.) (E. coli ligase buffer), 2 μl (20 μCi) of [α-³²P] dATP (>3000 ci/mmol), 12 μl of Nucleotide mix (10 mM each of dATP, dTTP, dGTP, and dCTP), and 288 μl of sterile water. The mixture was then cooled to 16° C., which was not subsequently exceeded. Next, 1.5 μl of E. coli ligase (10 U/μl) from Invitrogen (Carlsbad, Calif.), 2 μl of RNAse H (1.5 U/μl) from Stratagene (La Jolla, Calif.), and 11 μl of DNA polymerase I (10 U/μl) from Roche (Basel, Switzerland) were added. Then, the resulting mixture was incubated for 3 h at 16° C. The use of E. coli ligase dramatically increased the percentage of full-length cDNAs.

Fourth, to the main reaction mixture were added 210 μl of phenol (pH=8.0) and 210 μl of chloroform. After the mixture was mixed thoroughly and spun in a microfuge for 5 minutes, the aqueous phase was collected. To the remaining mixture was added 301 of 3 M sodium acetate (pH=5.0) followed by mixing. Then the mixture was charged with 600 μl of 2-propanol and incubated overnight at −20° C. Next, the mixture was spun at 13,000 rpm for 35 minutes at 4° C. After the mixture was washed with 70% ethanol, dried, and dissolved in 3711 of water, a 2 μl aliquot was removed for analysis of double-stranded cDNAs on a denaturing agarose gel. The analysis showed that double-stranded cDNA was also of high quality, with a maximum size of about 10 kb and an average size of about 2 kb. Such maximum and average sizes of double-stranded cDNAs indicated that the cDNA library contained a high percentage of full-length cDNA inserts.

Fifth, to the resulting double-stranded cDNA mixture (35 μl) was added 10 μl of 5× T4 DNA pol. buffer from Roche (Basel, Switzerland), 5 μl of dNTP mix (10 mM each dNTP), and 2 μl of T4 DNA polymerase (10 U/μl) from Roche (Basel, Switzerland). The mixture was incubated for 20 minutes at 37° C. to blunt-end the cDNA. Mixing helped to insure that a majority of the cDNA would be ligated with the EcoRI adapter.

Sixth, to the reaction mixture was added 250 μg of STE (100 mM NaCl/50 mM Tris pH 7.5/1mM EDTA), 150 μl of phenol (pH=8.0), and 150 μl of chloroform. The mixture was again mixed and spun in a microfuge for 5 minutes. An aqueous phase was collected. The remaining mixture was then charged with 15 μl of 3 M sodium acetate (pH=5.0). The mixture was then mixed and 300 μl of 2-propanol were added. The mixture was incubated for 3-5 h at −20° C. Then, the mixture was spun at 13,000 rpm for 35 minutes at 4° C. The mixture was washed with 70% ethanol, dried, and dissolved in 811 of EcoRI-adapter-mix from Stratagene (La Jolla, Calif.). Next, the resulting cDNA-EcoRI adapter mix was incubated for 1-2 h at 4° C., making sure that the cDNA was completely dissolved.

Seventh, to the cDNA-EcoRI adapter mix (8 μl) was added 1 μl of 10× Ligase Buffer from Stratagene (La Jolla, Calif.), 1 μl of 10 mM ATP from Stratagene (La Jolla, Calif.), and 0.8 μl of T4 DNA-ligase (5 Weiss Units/μl) from Roche (Basel, Switzerland). The mixture was incubated at 8° C. for 36 h. Notably, long ligation helped secure a high percentage (>80%) of cDNA to EcoRI adapter ligation.

Eighth, the ligase was heat-inactivated by incubating for 20 minutes at 70° C. Then, 2 μl of 10× Ligase Buffer from Stratagene (La Jolla, Calif.), 3 μl of 10 mM ATP from Stratagene (La Jolla, Calif.), 14 μl of water, and 2 μl of T4 polynucleotyde kinase (5 U/μl) from Stratagene (La Jolla, Calif.) were added. The mixture was incubated for 1 h at 37° C.

To heat inactivate phosphorylation of EcoRI adapters, the mixture was incubated for 20 minutes at 70° C. Then, 41 μl of XhoI Buffer from Stratagene (La Jolla, Calif.) and 4 μl of XhoI (40 U/μl) from Stratagene (La Jolla, Calif.) were added. The resulting mixture was then incubated for 3 h at 37° C.

Ninth, to the reaction mixture was added 230 μl of STE, 150 μl of phenol (pH=8.0), and 150 μl of chloroform. The mixture was mixed and then spun in a microfuge for 5 minutes. An aqueous phase was collected. To the remaining mixture was added 15 μl of 3.0 M sodium acetate (pH=5.0). Next, the mixture was mixed and 300 μl of 2-propanol were added. The mixture was then incubated overnight at −20° C. The mixture was then spun at 13,000 rpm for 35 minutes at 4° C., washed with 70% ethanol, dried, and dissolved in 2011 of water. Next, the mixture was incubated for 1-2 h at 4° C., insuring that the cDNA was completely dissolved. At this point, double-stranded cDNAs had 5′-end EcoRI sites (a majority of which were phosphorylated) and 3′-end XhoI-sites (all of which were phosphorylated).

Tenth, to increase the efficiency of ligation of cDNA into the expression vector pRK7, cDNAs were fractionated in 2.5% NuSieve agarose (a low-temperature melting agarose). Then, 1.5-4 kb, 4-6 kb, and >6 kb cDNA were eluted as separate zones. From this point, three separate cDNA libraries containing 1.5-4 kb, 4-6 kb, or >6 kb cDNA inserts were constructed. Final cDNAs (from each zone 1.5-4, 4-6, and >6 kb) were dissolved in 20 μl of water.

Eleventh, EcoRI-XhoI pRk7 expression vector was prepared in such a way that the XhoI-site was dephosphorylated and the EcoRI-site was phosphorylated. Moreover, linearized XhoI-EcoRI-pcDNA3 vector was purified twice by agarose fractionation. As a result, the expression vector preparation did not contain any contaminating sequence and was essentially pure. Next, linearized, ready-to-use vector was dissolved in 30 μl of water to yield a concentration of 0.2-0.3 μg/μl. The EcoRI-sites of the pRK7 vector were phosphorylated because not every EcoRI-site of cDNA contained a phosphate group, which is necessary for ligation.

Finally, double-stranded cDNAs were ligated into linearized expression vector pRK7 by mixing 1.5 ml of cDNA, 1 μl of 10× Ligase Buffer from Stratagene (La Jolla, Calif.), 1 μl of 10 mM ATP from Stratagene (La Jolla, Calif.), 0.5 μl (01-0.2 μg) of EcoRI-XhoI pRk7, 5 μl of water, and 0.8 μl of T4 DNA-ligase (5 Weiss Units/μl) from Roche (Basel, Switzerland). The mixture was then incubated for 14 h at 12° C., then 10 h at 16° C., then 1 h at 37° C., and then 3 days at 4° C.

The resulting ligation mix (0.5 μl) was used for transformation with 100 μl of XL-10 Ultracompetent cells from Stratagene (La Jolla, Calif.) at 109 colonies per mg DNA, according to a Stratagene protocol. A 10 μl ligation mixture yielded 100,000 colonies for zone 1.5-4 kb; 20,000 colonies for zone 4-6 kb, and 40,000 colonies for zone >6 kb. The final enriched library contained 30 pools (zone 1.5-4 kb) with 500-800 colonies each, 20 pools (zone 4-6 kb) with 500-800 colonies each, and 20 pools (zone >6 kb) with 500-800 colonies each.

### Example 3

Both a conventional cDNA expression library from total polyA⁺ RNA and an enriched cDNA expression library from membrane-bound polysomal mRNA were constructed from rat trigeminal ganglia (TG). To do so, 20 rats were sacrificed to construct a conventional expression cDNA library and 80 rats were sacrificed to generate an enriched expression cDNA library. Total RNA for the conventional expression cDNA library was isolated as described in Chomczynski and Sacchi, Anal Biochem (1987) 162(1):156-159. Isolation of membrane-bound polysomal RNA for the enriched cDNA expression library was as described in Example 1. Poly A⁺ RNA for both libraries was isolated according to Aviv and Leder, Proc Natl Acad Sci USA (1972) 69(6):1408-1412, which is incorporated herein by reference in its entirety for the method.

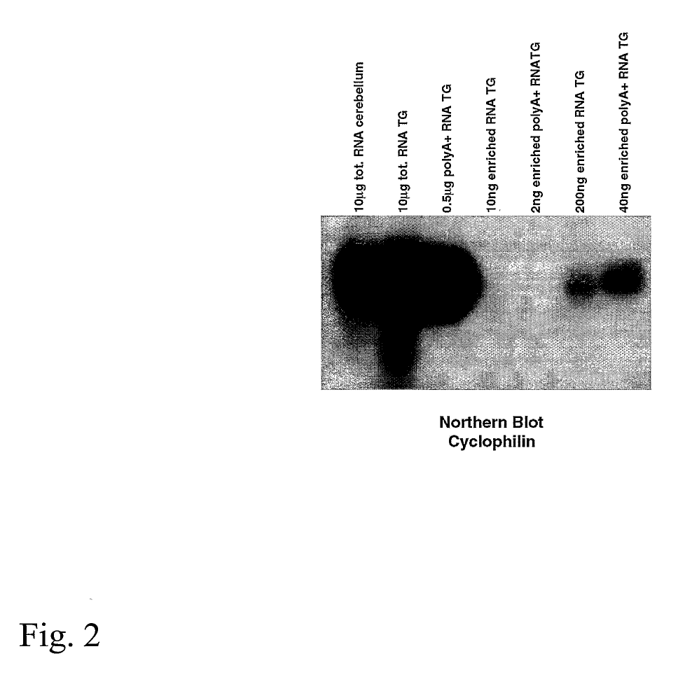

The purification level for each isolation and purification procedure was defined using Northern blot hybridization against a cyclophilin probe (FIG. 2). Amounts and isolation methods of loaded RNA are given in FIG. 2. The results indicated that a 95-97% purification level was achieved when the enrichment procedure described in Example 1 was used. Also, the enrichment procedure for membrane-bound polysomal mRNA yielded a small amount of mRNA; that is, 3-4 μg from approximately 2.4 mg of TG tissue. Generally, the amount for poly A⁺ RNA isolated by conventional methods is 15-20 times greater than a yield of the membrane-bound polysomal mRNA.

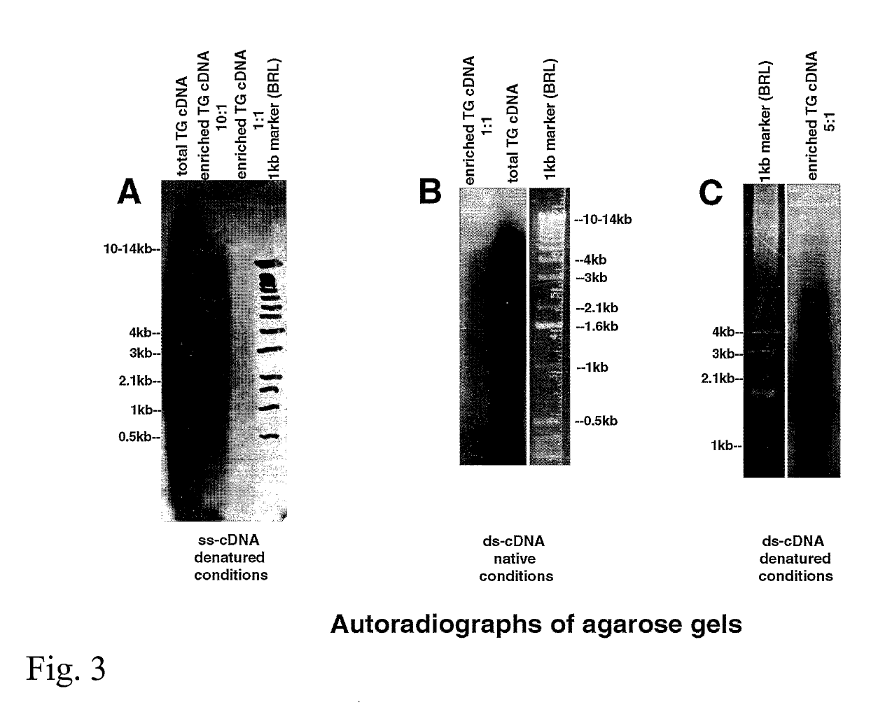

The enriched and conventional cDNA expression libraries were constructed as described in Example 2. FIG. 3 shows the quality of synthesized enriched or total (i.e., conventional) TG cDNA at the different stages of cDNA library generation. Auto-radiographic images are of agarose gels run under a variety of conditions. The gel electrophoresis shown in FIG. 3A was run after step 1 of Example 2. The gel electrophoresis in FIGS. 3B and 3C were run after step 4 of Example 2. FIGS. 3A and 3B show that sizes of the single-stranded as well as double-strand cDNAs reached 10-14 kb, whereas an uninterrupted synthesis of the double-stranded cDNAs reached up to 6-7 kb (see FIG. 3C). The length of uninterrupted synthesis directly affected the efficiency of full-length cDNA production in the library (i.e., number of full-length clones in the library per 1 μg of cDNA). Only full-length cDNA clones, which contain the start codon (ATG), could participate in protein synthesis. Synthesized proteins were used for functional tests.

Analysis of Constructed cDNA Expression Libraries:

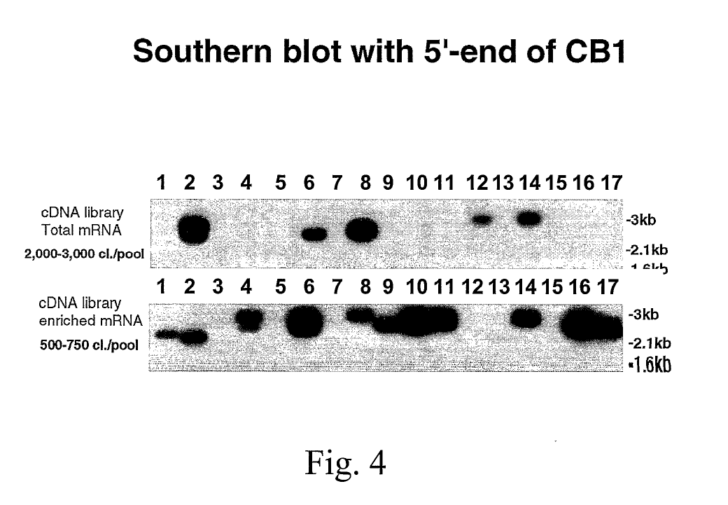

The enriched and conventional cDNA expression libraries were divided into pools. The number of clones in the pools is indicated in FIG. 4. Also obtained were hybridized cDNA pools (cut by EcoRI and XhoI restriction enzymes) with a 5′-end of the cannabinoid type 1 (CB1) receptor probe. The CB1 receptor mRNA is approximately 6 kb. According to GenBank, the rat CB1 receptor cDNA has the 2.5 kb-EcoRI-EcoRI-fragment at the 5′-end, which contains the start (ATG) codon and approximately 0.41kb of the 5′-untranslated region. Therefore, hybridized fragments longer than 2.1-2.2 kb indicated the presence of a full-length translatable clone. In the conventional cDNA expression library, the CB 1 receptor cDNA was present in 5 of the 17 pools, and pool-2 contained two CB1 receptor clones (see the upper panel in FIG. 4). In the enriched cDNA expression library, the CB1 receptor cDNA was present in 11 of the 17 pools, and pools-4, -6, -10, -11, -16 and -17 contained at least two CB1 receptor clones (see the lower panel in FIG. 4). Altogether, the enriched cDNA expression library had 3-times more CB1 receptor-positive clones than the conventional cDNA expression library (i.e., 17 vs. 6), despite the fact that the pools of the enriched cDNA expression library had 4-times less overall clones than the pools of the conventional cDNA expression library. This result indicates that a 12-fold enrichment for 6 kb-CB1 receptor cDNA was achieved.

Throughout this application, various publications, patents, and/or patent applications are referenced in order to more fully describe the state of the art to which this invention pertains. The disclosures of these publications, patents, and/or patent applications are herein incorporated by reference in their entireties, and for the subject matter for which they are specifically referenced in the same or a prior sentence, to the same extent as if each independent publication, patent, and/or patent application was specifically and individually indicated to be incorporated by reference.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only.

What is claimed is:

1. A method of making a cDNA expression library enriched for cDNAs that encode secretory or membrane-bound proteins, comprising the steps of:
a. isolating membrane bound polysomal RNA from a selected population of cells;
b. isolating polyadenylated RNA from the isolated membrane-bound polysomal RNA from step (a);
c. constructing a cDNA expression library from the isolated polyadenylated
RNA from step (b),
wherein the cDNA expression library comprises more than 90% cDNAs that encode secretory and membrane-bound proteins.

2. The method of claim 1, wherein the membrane bound polysomal RNA of step (a) is isolated by homogenizing the selected population of cells in a high salt buffer to form a homogenate, wherein the homogenate is centrifuged to form a supernatant, and wherein the supernatant is fractionated by centrifugation through a sucrose gradient to isolate the membrane-bound polysomal RNA.

3. The method of claim 2, wherein the high salt buffer lacks a detergent additive.

4. The method of claim 2, wherein the supernatant is fractionated through an inverted ultra-centrifugation step.

5. The method of claim 1, wherein the polyadenylated RNA is isolated by an oligo-dT cellulose affinity purification step.

6. The method of claim 1, wherein the cDNA expression library is constructed in a vector selected from the group consisting of pcDNA3 and pRK7.

7. The method of claim 6, wherein the expression vector is pRK7.

8. The method of claim 1, wherein the selected population of cells comprises neurons.

9. The method of claim 8, wherein the neurons are sensory neurons.

10. The method of claim 8, wherein the sensory neurons are dorsal root ganglion neurons.

11. The method of claim 8, wherein the sensory neurons are cranial nerve sensory ganglion neurons.

12. The method of claim 11, wherein the cranial nerve sensory ganglion neurons are trigeminal ganglion neurons.

13. A cDNA expression library made by the method of claim 1.

14. A method of screening for selected membrane-bound proteins or secretory proteins, comprising the steps of:
a. contacting the proteins expressed by the cDNA expression library of claim 13 with a marker that binds the selected membrane-bound proteins or secretory proteins;
b. detecting the bound marker, the bound marker indicating the presence of selected membrane-bound proteins or secretory proteins.

15. A method of screening for cDNAs that encode selected membrane-bound proteins, comprising the steps of:
a. contacting the cDNA expression library of claim 13 with a nucleic acid that selectively hybridizes under stringent condition with cDNA that encodes selected membrane-bound proteins;
b. detecting the hybridizing cDNA, the hybridizing cDNA indicating the presence of the cDNAs that encode the selected membrane-bound proteins.

16. A method of screening for agents that modulate selected membrane-bound proteins or secretory proteins, comprising:
a. contacting the agent to be screened with membrane bound proteins or secretory proteins selected by the method of claim 14;
b. detecting an increase or decrease in a selected function of the membrane-bound or secretory proteins as compared to the selected membrane-bound proteins or secretory proteins in the absence of the agent, an increase or decrease in function indicating an agent that modulates the selected membrane-bound or secretory proteins.

17. The method of claim 16, wherein the selected function of the membrane-bound or secretory proteins is specific binding to an agonist, antagonist, modulator, or co-factor.

18. The method of claim 16, wherein the selected function of the membrane-bound or secretory proteins is specific activity.

19. The method of claim 18, wherein the specific activity is channel activity.

20. The method of claim 18, wherein the specific activity is metabotropic activity.

21. The method of claim 18, wherein the specific activity is enzyme activity.

22. The method of claim 16, wherein the selected function of the membrane-bound or secretory proteins is specific binding to a co-factor.

23. The method of claim 16, wherein the selected function of the membrane-bound or secretory proteins is intracellular signaling.

24. The method of claim 16, wherein the contacting step comprises contacting a cell that expresses the membrane bound proteins or secretory proteins.

25. A method of screening for agents that modulate expression of selected membrane-bound proteins or secretory proteins comprising:
a. contacting the agent to be screened with a test cell that expresses the selected membrane-bound or secretory proteins encoded by the cDNAs identified by the method of claim 15;
b. detecting an increase or decrease in expression of the membrane-bound or secretory proteins as compared to the selected membrane-bound proteins or secretory proteins in the absence of the agent, an increase or decrease in expression indicating an agent that modulates expression of the selected membrane-bound or secretory proteins.

26. A method of screening for agents that modulate expression of selected membrane-bound proteins or secretory proteins, comprising:
a. contacting the agent to be screened with a test cell that expresses the selected membrane-bound or secretory proteins identified by the method of claim 14;
b. detecting an increase or decrease in expression of the membrane-bound or secretory proteins in the test cell as compared to expression of the selected membrane-bound proteins or secretory proteins in a control cell in the absence of the agent, an increase or decrease in expression in the test cell indicating an agent that modulates expression of the selected membrane-bound or secretory proteins.

27. A method of screening for agents that modulate expression of selected membrane-bound proteins or secretory proteins, comprising:
a. contacting the agent to be screened with a test cell that expresses the selected membrane-bound or secretory proteins;
b. comparing nucleic acid expression by the cell with the cDNAs identified by the method of claim 15, an increase or decrease in expression by the test cell as compared to the expression by a control cell in the absence of the agent indicating an agent that modulates expression of the selected membrane-bound or secretory protein.

28. A microarray comprising the cDNAs from the expression library of claim 11.

29. A cDNA expression library comprising more than 90% cDNAs that encode secretory and membrane-bound proteins.

30. A method of screening a microarray of nucleic acids, comprising contacting the nucleic acids of the microarray with the cDNAs of the cDNA expression library of claim 29.
